SEQUENCE LISTING (20 sequences)

SEQ ID NO: 1 - protein, 8 aa, Homo sapiens
  MOD_RES 1: note = ACETYLATION
  MOD_RES 8: note = AMIDATION
KPSSPPEE

SEQ ID NO: 2 - protein, 8 aa, Homo sapiens
  MOD_RES 1: note = ACETYLATION
  MOD_RES 8: note = AMIDATION
NASAPPEE

SEQ ID NO: 3 - protein, 14 aa, Homo sapiens
QETWFQNGWQGKNP

SEQ ID NO: 4 - protein, 14 aa, Homo sapiens
KEKWFENEWQGKNP

SEQ ID NO: 5 - protein, 14 aa, Homo sapiens
KEQWFGNRWHEGYR

SEQ ID NO: 6 - protein, 24 aa, Homo sapiens
QIRQQPRDPPTETLELEVSPDPAS

SEQ ID NO: 7 - protein, 8 aa, synthetic construct
  REGION 1..8: note = Mutated CD-44 modulating polypeptide
  MOD_RES 1: note = ACETYLATION
  MOD_RES 8: note = AMIDATION
APSSPPEE

SEQ ID NO: 8 - protein, 8 aa, synthetic construct
  REGION 1..8: note = Mutated CD-44 modulating polypeptide
  MOD_RES 1: note = ACETYLATION
  MOD_RES 8: note = AMIDATION
KASSPPEE

SEQ ID NO: 9 - protein, 8 aa, synthetic construct
  REGION 1..8: note = Mutated CD-44 modulating polypeptide
  MOD_RES 1: note = ACETYLATION
  MOD_RES 8: note = AMIDATION
KPSSAPEE

SEQ ID NO: 10 - protein, 8 aa, synthetic construct
  REGION 1..8: note = Mutated CD-44 modulating polypeptide
  MOD_RES 1: note = ACETYLATION
  MOD_RES 8: note = AMIDATION
KPSSPAEE

SEQ ID NO: 11 - protein, 8 aa, synthetic construct
  REGION 1..8: note = Mutated CD-44 modulating polypeptide
  MOD_RES 1: note = ACETYLATION
  MOD_RES 8: note = AMIDATION
KASSPAEE

SEQ ID NO: 12 - protein, 8 aa, synthetic construct
  REGION 1..8: note = Mutated CD-44 modulating polypeptide
  MOD_RES 1: note = ACETYLATION
  MOD_RES 8: note = AMIDATION
KPSSAAEE

SEQ ID NO: 13 - protein, 8 aa, synthetic construct
  REGION 1..8: note = Mutated CD-44 modulating polypeptide
  MOD_RES 1: note = ACETYLATION
  MOD_RES 8: note = AMIDATION
KPASPPEE

SEQ ID NO: 14 - protein, 8 aa, synthetic construct
  REGION 1..8: note = Mutated CD-44 modulating polypeptide
  MOD_RES 1: note = ACETYLATION
  MOD_RES 8: note = AMIDATION
KPSAPPEE

SEQ ID NO: 15 - protein, 8 aa, synthetic construct
  REGION 1..8: note = Mutated CD-44 modulating polypeptide
  MOD_RES 1: note = ACETYLATION
  MOD_RES 8: note = AMIDATION
KPAAPPEE

SEQ ID NO: 16 - protein, 8 aa, synthetic construct
  REGION 1..8: note = Mutated CD-44 modulating polypeptide
  MOD_RES 1: note = ACETYLATION
  MOD_RES 8: note = AMIDATION
KPSSPPAE

SEQ ID NO: 17 - protein, 8 aa, synthetic construct
  REGION 1..8: note = Mutated CD-44 modulating polypeptide
  MOD_RES 1: note = ACETYLATION
  MOD_RES 8: note = AMIDATION
KPSSPPEA

SEQ ID NO: 18 - protein, 8 aa, synthetic construct
  REGION 1..8: note = Mutated CD-44 modulating polypeptide
  MOD_RES 1: note = ACETYLATION
  MOD_RES 8: note = AMIDATION
KPSSPPAA

SEQ ID NO: 19 - protein, 8 aa, synthetic construct
  REGION 1..8: note = Mutated CD-44 modulating polypeptide
  MOD_RES 1: note = ACETYLATION
  MOD_RES 8: note = AMIDATION
APSSPPAE

SEQ ID NO: 20 - protein, 8 aa, synthetic construct
  REGION 1..8: note = Mutated CD-44 modulating polypeptide
  MOD_RES 1: note = ACETYLATION
  MOD_RES 8: note = AMIDATION
KPSAAPEE

(19) United States
(12) Patent Application Publication
(10) Pub. No.: US 2023/0183290 A1
(43) Pub. Date: Jun. 15, 2023

(54) METHODS FOR TREATING CANCER USING SPL-108 POLYPEPTIDE BASED ON TP53 MUTATIONAL STATUS

(71) Applicant: Splash Pharmaceuticals, Inc., San Diego, CA (US)

(72) Inventors: Malcolm Finlayson, San Diego, CA (US); David Nelson, San Diego, CA (US)

(21) Appl. No.: 18/055,227

(22) Filed: Nov. 14, 2022

Related U.S. Application Data
(63) Continuation-in-part of application No. PCT/US21/32043, filed on May 12, 2021.
Application No. PCT/US21/32043 is a continuation-in-part of application No. PCT/US21/50696, filed on Sep. 16, 2021.
Application No. PCT/US21/50696 is a continuation-in-part of application No. PCT/US21/32043, filed on May 12, 2021.
(60) Provisional application No. 63/023,591, filed on May 12, 2020.
(60) Provisional application No. 63/079,404, filed on Sep. 16, 2020.

(51) Int. Cl. C07K 7/06; A61P 35/00; A61K 31/337

(52) U.S. Cl. CPC C07K 7/06; A61P 35/00; A61K 31/337

(57) ABSTRACT

The present invention relates to methods of treatment for cancer using SPL-108 polypeptide (also known as A6 peptide or A6, a CD44 modulating peptide; FIG. 1; SEQ ID NO:1) based on the TP53 status of the cancer. In particular, this invention relates to such methods of treatment by using TP53 mutational status to guide the administration of an SPL-108 polypeptide or a variant thereof alone or in combination with a standard-of-care treatment for patients with cancer. TP53 mutational status is used to select patients that may or may not respond to SPL-108 polypeptide or a variant thereof; specific mutations TP53 predict the response of patients including progressive disease, stable disease, and/or partial/complete response to SPL-108. Such methods of the present invention are used to treat cancer to alleviate and/or prevent symptoms of cancer, and/or to avoid side effects commonly associated with treating cancer.

### CROSS-REFERENCES TO RELATED APPLICATIONS

This application is a Continuation-In-Part and claims benefit of PCT/US21/32043 filed May 12, 2021, which claims benefit of U.S. Provisional Patent Application No. 63/023,591 filed May 12, 2020, the specification(s) of which is/are incorporated herein in their entirety by reference.

This application is also a Continuation-In-Part and claims benefit of PCT/US21/50696 filed Sep. 16, 2021, which claims benefit of PCT/US21/32043 filed May 12, 2021 and U.S. Provisional Patent Application No. 63/079,404 filed Sep. 16, 2020, the specification(s) of which is/are incorporated herein in their entirety by reference.

### REFERENCE TO AN ELECTRONIC SEQUENCE LISTING

The contents of the electronic sequence listing (SPLASH_20_01_PCT_CIP_Sequence_Listing.xml; Size: 35,000 bytes; and Date of Creation: Jan. 10, 2023) is herein incorporated by reference in its entirety.

### FIELD OF THE INVENTION

The present invention relates to methods of treatment for cancer using SPL-108 polypeptide (also known as A6 peptide or A6, a CD44 modulating peptide; FIG. 1; SEQ ID NO:1) based on the TP53 status of a cancer. In particular, this invention relates to such methods of treatment by using TP53 mutational status to guide the administration of an SPL-108 polypeptide or a variant thereof alone or in combination with a standard-of-care treatment for patients with cancer. TP53 mutational status is used to select patients that may or may not respond to SPL-108 polypeptide or a variant thereof; specific mutations TP53 predict the response of patients including progressive disease, stable disease, and/or partial/complete response to SPL-108. Such methods of the present invention are used to treat cancer to alleviate and/or prevent symptoms of cancer, and/or to avoid side effects commonly associated with treating cancer.

### BACKGROUND OF THE INVENTION

Mortality due to cancer is generally the result of metastasis of the primary tumor. Recurrence at distant sites following first-line therapy continues to be a major challenge. As a result, drugs that inhibit the metastatic process are of great interest. Metastasis and recurrence have been linked to a subpopulation of highly invasive tumorigenic cells that are characterized by the expression of CD44. In addition, alterations mutations in TP53, a tumor suppressor, have been associated with a poor response to chemotherapy, hormonotherapy, or radiotherapy. There is a need for anti-cancer agents that can inhibit metastasis in cancer patients and produce good response rates. Provided herein are solutions to these problems and other problems in the art

### BRIEF SUMMARY OF THE INVENTION

It is an objective of the present invention to provide methods of using SPL-108 (or A6) polypeptide, a variant thereof, or a CD44-modulating peptide alone or in combination with other agents that allow for effectively treating cancer alleviating symptoms of cancer, and avoiding side effects or allergic reactions associated with treating cancer; the use of SPL-108 is based on the cancer mutational status of TP53, as specified in the independent claims. Embodiments of the invention are given in the dependent claims. Embodiments of the present invention can be freely combined with each other if they are not mutually exclusive.

One of the unique and inventive technical features of the present invention is the use of the mutational status of TP53 and a targeted peptide (e.g., for CD44 expressing cells) in the treatment of cancer. Without wishing to limit the invention to any theory or mechanism, it is believed that the technical feature of the present invention advantageously provides for a method that allows for the prediction of how a patient will respond to the targeted peptide. Additionally, the targeting peptide alleviates symptoms of cancer while avoiding common side effects associated with treating cancer. None of the presently known prior references or work has the unique inventive technical feature of the present invention.

Furthermore, the inventive technical features of the present invention contributed to a surprising result. For example, previous studies have shown that TP53 mutations are not associated with overall survival. Additionally, analysis of The Cancer Genome Atlas (TCGA) database compared 1) overall survival in patients with p53 mutations resulting in truncations compared to p53 mutations not resulting in truncations, 2) p53 mutations resulting in truncations compared to p53 wild type, and finally, 3) those with any p53 mutation compared to p53 wild type, and was not able to find any survival differences in any of these three comparisons. However, the present invention is surprisingly able to utilize the mutational status of TP53 to predict the ability of the presently claimed SPL-108 to restore p53 activity for the treatment of cancer.

In some embodiments, the present invention features a method of treating a patient with a polypeptide comprising Ac-KPSSPPEE-NH2 (SEQ ID NO:1), an active variant thereof, or a CD44-modulating peptide, wherein the patient has cancer. In some embodiments, the method comprises determining the TP53 mutational status. In some embodiments, TP53 mutational status is determined by obtaining or having obtained a biological sample from the patient and performing or having performed a genotyping assay on the biological sample to determine the TP53 mutational status. In some embodiments, the method comprises administering a polypeptide comprising Ac-KPSSPPEE-NH2 (SEQ ID NO:1), an active variant thereof, or a CD44-modulating peptide, to a patient if the TP53 mutation status predicts a p53 loss of activity.

In other embodiments, the present invention features a method of determining the effectiveness of a polypeptide comprising Ac-KPSSPPEE-NH2 (SEQ ID NO:1), an active variant thereof, or a CD44-modulating peptide to treat cancer in a patient. In some embodiments, the method comprises determining a TP53 mutational status. In some embodiments, the TP53 mutational status is determined by obtaining or having obtained a biological sample from the patient and performing or having performed a genotyping assay on the biological sample to determine the TP53 mutational status. In some embodiments, the method comprises determining the effectiveness of the polypeptide to treat cancer in the patient based on the TP53 mutational status. In some embodiments, if the TP53 mutational status predicts a p53 loss of activity, the polypeptide is determined to be effective at treating cancer in the patient, and the polypeptide is administered to the patient. In other embodiments, if the TP53 mutational status predicts no tumor p53 expression or TP53 loss of function, the polypeptide is determined to not be effective at treating cancer in the patient, and the polypeptide is not administered to the patient

In some embodiments, the present invention also features a composition for use in treating a patient with cancer. In some embodiments, the composition comprises a polypeptide comprising Ac-KPSSPPEE-NH2 (SEQ ID NO:1), an active variant thereof, or a CD44-modulating peptide. In some embodiments, a TP53 mutational status of the patient is used to determine the effectiveness of the composition. In other embodiments, the composition is administered to the cancer patient if the TP53 mutation status predicts a loss of p53 activity.

In some embodiments, the present invention features methods of treating a cancer, inhibiting metastasis of a cancer, treating a resistant or refractory cancer, and/or restoring the anti-cancer activity of radiation therapy in a patient in need thereof. The present invention further features methods of avoiding side effects or allergic reactions associated with treating cancer and/or alleviating symptoms of cancer. Non-limiting examples of cancers that may be treated using the present invention include ovarian cancer, breast cancer, colorectal cancer, prostate cancer, head and neck cancer, endometrial cancer, primary peritoneal cancer, liver cancer, glioblastoma, or a combination thereof. In preferred embodiments, these methods comprise first determining TP53 mutational status in a biological sample (e.g., tissue, cells, blood, saliva, cerebrospinal fluid CSF) obtained from the patient with cancer. The patient is then screened and/or selected to be eligible to receive a treatment comprising a polypeptide, such as Ac-KPSSPPEE-NH2 (SEQ ID NO: 1), an active variant thereof, or a CD44-modulating peptide, based on the determined TP53 mutational status in a biological sample obtained from the patient. Then during a first period, the treatment comprising the polypeptide is administered to the screened or selected patient afflicted with cancer at an effective daily dose over a period of time sufficient to effectively treat the cancer. Without wishing to limit the present invention to any particular theory or mechanism, the TP53 mutational status predicts the response of the patient to SPL-108 an active variant thereof, or a CD44-modulating peptide treatment.

Non-limiting examples of treatment responses comprise complete response, partial response, stable disease, and/or disease progression as defined in Response Evaluation Criteria in Solid Tumors (RECIST) Criteria 1.1. Non-limiting examples of TP53 mutational status comprise R342* (* mutation in oligomerization domain), D259V (non-functional protein), R175H, R273C, R213* (*: truncated/nonsense), G105fs* (*: truncated/frameshift), E286Q, and/or C238W mutations in TP53, and/or TP53 wildtype (WT). In some embodiments, TP53 mutational status predicts partial response, R342* TP53 mutational status predicts partial response, D259V TP53 mutational status predicts progressive disease, R175H TP53 mutational status predicts stable disease, R273C TP53 mutational status predicts partial response, R213* (*: truncated/nonsense) TP53 mutational status predicts progressive disease, G105fs* (*: truncated/frameshift) TP53 mutational status predicts progressive disease, E286Q TP53 mutational status predicts stable disease, and/or C238W TP 53 mutational status predicts partial response. In other embodiments, WT TP53 status predicts stable disease.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 illustrates that the polypeptide of SEQ ID NO:1 shares sequence homology with a portion of the Link-Domain of CD44 (CD44 amino acid residues 120-NASAPPEE-127)

FIG. 2 shows the frequency of select TP53 mutations

### DETAILED DESCRIPTION OF THE INVENTION

All patents, applications, published applications, and other publications are incorporated by reference in their entirety and for all purposes. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices, and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit, the invention described herein.

As used herein, the term “TP53” refers to tumor protein p53, also known as p53, cellular tumor antigen p53 (UniProt name), the Guardian of the Genome, phosphoprotein p53, tumor suppressor p53, antigen NY-CO-13, or transformation-related protein 53 (TRP53), is any isoform of a protein encoded by homologous genes in various organisms, such as TP53 (humans) and Trp53 (mice). This homolog is crucial in multicellular vertebrates, where it prevents cancer formation, and thus functions as a tumor suppressor. TP53 has a role in conserving stability by preventing genome mutation. TP53 is classified as a tumor suppressor gene.

p53 is a natural tumor suppressor activated by DNA damage that inhibits tumorigenesis and is widely known to be mutated in numerous cancers. Alterations in p53 that result in no p53 tumor-suppressing activity lead to metastatic disease. Additionally, p53 alterations are associated with resistant and refractory disease. Restoration of normal p53 activity counters this by repressing the MDR1 (i.e., multidrug resistance (MDR1) gene) pathway and restores chemo-sensitivity to resistant cells.

As used herein, the terms “polypeptide” and “protein” are used interchangeably and refer to any molecule that includes at least 2 or more amino acids.

As used herein, “administering” and the like refer to the act physically delivering a composition or other therapy (e.g. radiation therapy) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. Radiation therapy can be administered using techniques described herein, including, for example, external beam radiation or brachytherapy. When a disease, disorder, or condition, or a symptom thereof, is being treated, administration of the substance typically occurs after the onset of disease, disorder, or condition or symptoms thereof. When a disease, disorder, or condition, or symptoms thereof, are being prevented, administration of the substance typically occurs before the onset of the disease, disorder, or condition, or symptoms thereof.

The term “coadministration” refers to the administration of two or more agents (e.g., a polypeptide described herein and another active agent such as an anti-cancer agent or other therapy (e.g. radiation therapy) described herein). The timing of coadministration depends in part on the combination and compositions or other therapies administered and can include administration at the same time, just prior to, or just after the administration of one or more additional therapies, for example, cancer therapies such as chemotherapy, hormonal therapy, radiotherapy, or immunotherapy. Coadministration is meant to include simultaneous or sequential administration of a composition or therapy individually or in combination (more than one polypeptide described herein or an anti-cancer agent described herein or radiation therapy as described herein). Coadministration can include the administration of two or more agents where the agents are optionally combined with other active substances (e.g., to reduce metabolic degradation). The polypeptides, anti-cancer agents, and radiation therapies described herein can be used in combination with one another, with other active agents known to be useful in treating a disease associated with cells expressing a particular kinase as described herein, or with adjunctive agents that cannot be effective alone but can contribute to the efficacy of the active agent.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human.

The terms “treating” or “treatment” refer to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “cancer” refers to any physiological condition in mammals characterized by unregulated cell growth. Cancers described herein include solid tumors and hematological (blood) cancers. A “hematological cancer” refers to any blood-borne cancer and includes, for example, myelomas, lymphomas, and leukemias. A “solid tumor” or “tumor” refers to a lesion and neoplastic cell growth and proliferation, whether malignant or benign and all precancerous and cancerous cells and tissues resulting in abnormal tissue growth. “Neoplastic,” as used herein, refers to any form of dysregulated or unregulated cell growth, whether malignant or benign, resulting in abnormal tissue growth.

An improvement in the cancer or cancer-related disease can be characterized as a complete or partial response. Complete response refers to an absence of clinically detectable disease with normalization of any previously abnormal radiographic studies, bone marrow, and cerebrospinal fluid (CSF), or abnormal monoclonal protein measurements. Partial response refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

A refractory, resistant, or persistent cancer refers to a circumstance where patients, even after intensive treatment, have residual cancer cells (e.g., leukemia cells, lymphoma cells, circulating tumor cells, or cancer stem cells) in their lymphatic system, blood, and/or blood-forming tissues (e.g., marrow).

The terms “manage,” “managing,” and “management” refer to preventing or slowing the progression, spread, or worsening of a disease or disorder, or of one or more symptoms thereof. In certain cases, the beneficial effects that a subject derives from a prophylactic or therapeutic agent do not result in a cure of the disease or disorder.

The term “preventing” refers to the treatment with or administration of a polypeptide or agent (e.g. anti-cancer agent described herein) provided herein, with or without other additional active agents (e.g. an anti-cancer agent), prior to the onset of symptoms, particularly to patients at risk of cancer and/or other disorders described herein. The term also refers to coadministration of a polypeptide with other therapies including radiation therapies as described herein. It should be understood that the polypeptides described herein can be coadministered with one or more anti-cancer agents and radiation therapies described herein. The term prevention includes the inhibition or reduction of a symptom of the particular disease, as well as a reduced incidence of a symptom of the particular disease (e.g. by comparison to historical data for a given subject, or population data for similar subjects). Patients with familial history of a disease, in particular, are candidates for preventive regimens in certain embodiments. In addition, patients who have a history of recurring symptoms are also potential candidates for the prevention. In this regard, the term “prevention” may be interchangeably used with the term “prophylactic treatment.”

A prophylactically effective amount of a polypeptide or agent (e.g. an anti-cancer agent described herein) means an amount of therapeutic agent, alone or in combination with other agents, which provides a prophylactic benefit in the inhibition or reduced incidence of a symptom of a disease or recurrence of a disease. The term also refers to coadministration of a polypeptide described herein with other therapies including radiation therapies as described herein. The term prophylactically effective amount can encompass an amount that improves overall prophylaxis or enhances the prophylactic efficacy of another prophylactic agent.

The term “effective amount” as used herein refers to the amount of a therapy (e.g., a composition or radiation therapy provided herein) that is sufficient to reduce and/or ameliorate the severity and/or duration of a given disease, disorder, or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of a polypeptide described herein, an anti-cancer agent described herein, or a radiation therapy described herein is an amount sufficient to provide a therapeutic benefit in the treatment or management of a cancer or to delay or minimize one or more symptoms associated with the presence of the cancer. A therapeutically effective amount of a polypeptide described herein, an anti-cancer agent described herein, or a radiation therapy described herein means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

A therapy is any protocol, method, and/or agent that can be used in the prevention, management, treatment, and/or amelioration of a given disease, disorder, or condition. In certain embodiments, the terms “therapies” and “therapy” refer to a drug therapy, biological therapy, supportive therapy, radiation therapy, and/or other therapies useful in the prevention, management, treatment, and/or amelioration of a given disease, disorder or condition known to one of skill in the art such as medical personnel.

A regimen is a protocol for dosing and timing the administration of one or more therapies (e.g., combinations described herein, another active agent such as for example an anti-cancer agent described herein, or a radiation therapy described herein) for treating a disease, disorder, or condition described herein. A regimen can include periods of active administration and periods of rest as known in the art. Active administration periods include administration of combinations and compositions described herein and the duration of time of efficacy of such combinations, compositions, and radiation therapies. Rest periods of regimens described herein include a period of time in which no agent (e.g., a polypeptide described herein or an anti-cancer agent described herein) is actively administered, and in certain instances, includes time periods where the efficacy of such agents can be minimal. Rest periods of regimens described herein can include a period of time in which no radiation therapy is actively administered. Combination of active administration and rest in regimens described herein can increase the efficacy and/or duration of administration of the combinations and compositions described herein.

The term “pharmaceutically acceptable” as used herein refers to physiologically acceptable compounds, agents, or ingredients recognized by a regulatory agency of the Federal or state government, or another governmental agency with authorization for such approval, or and an agent listed in the U.S. Pharmacopeia, European Pharmacopeia or other generally recognized Pharmacopeia for use in animals, and more particularly in humans.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, sucrose, glucose, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms binder, filler, disintegrant, and lubricant are used in accordance with the plain and ordinary meaning within the art.

In certain embodiments, a pharmaceutically acceptable excipient may be incompatible (e.g., cross-reacts) with other excipients or active agents described herein. In some embodiments, magnesium stearate, croscarmellose sodium, lactose, excipients comprising Mg, Ca, K, Li, or Nucleic acid, acesulfame potassium, ammonium alginate, calcium acetate, calcium alginate, calcium carbonate, calcium chloride, calcium lactate, calcium phosphate, calcium silicate, calcium stearate, calcium sulfate, carboxymethylcellulose calcium, carboxymethylcellulose sodium, docusate sodium, glycine, kaolin, magnesium aluminum silicate, magnesium carbonate, magnesium oxide, magnesium silicate, magnesium trisilicate, polacrilin potassium, polymethacrylates, potassium alginate, potassium benzoate, potassium bicarbonate, potassium chloride, potassium citrate, sodium alginate, sodium benzoate, sodium chloride, sodium lauryl sulfate, sodium starch glycolate, sodium stearyl fumarate, sulfobutylether beta-cyclodextrin, sodium stearate, talc, or zinc stearate are incompatible in the dosage forms described herein.

The term “anti-cancer agent” is used in accordance with its plain ordinary meaning and refers to a composition having anti-neoplastic properties or the ability to inhibit the growth or proliferation of cells. In certain embodiments, an anti-cancer agent is a chemotherapeutic agent. In certain embodiments, an anti-cancer agent is an agent identified herein having utility in methods of treating cancer. In certain embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” or “cancer therapy” refers to a therapy or regimen that includes administration of a combination, chemotherapeutic, or anti-cancer agent described herein.

The term “radiation therapy” is used in accordance with its plain ordinary meaning and refers to the medical use of radiation in the treatment of cancer. Preferably, the medical use of radiation in the treatment of cancer results in the killing of cancer cells in the subject. A variety of radiation therapies can be used in accordance with the present disclosure, examples of which are provided herein.

A “CD44-modulating polypeptide” refers to a polypeptide that binds to CD44 and modulates its activity (e.g., signaling activity). A CD44-modulating polypeptide can be a polypeptide sequence described herein or, in some embodiments, an antibody that specifically binds to CD44 and inhibits its downstream signaling activity. In one embodiment, a CD44-modulating polypeptide can be a polypeptide sequence described herein or, in some embodiments, an antibody that disrupts or inhibits the signaling activity of a CD44 dependent co-receptor. In certain instances, the CD44 dependent co-receptor is a receptor tyrosine kinase (RTK) such as, for example, Met, Ran, or VEGFR. In still another embodiment a CD44-modulating polypeptide can be a polypeptide sequence described herein or, in some embodiments, an antibody that disrupts CD44 co-receptor function or association of a CD44 co-receptor with CD44 or another signaling protein. In one embodiment, a CD44-modulating polypeptide described herein binds to CD44 and inhibits CD44 signaling activity or association with one or more ATP-binding cassette transporters (ABC transporters). The ABC transporter can be a multidrug-resistant protein (e.g., MDR1). In certain embodiments, CD44 levels can be elevated upon radiation therapy. Exemplary CD-44 modulating polypeptides include polypeptides having homology to the CD44-v6 region of human CD44. Such peptides can include substitution variants, addition variants, or chemical derivatives thereof including peptidomimetics. In one embodiment, the CD44-modulating polypeptide described herein is a polypeptide having the amino acid sequence of Ac-KPSSPPEE-NH₂ (SEQ ID NO:1), Ac-NASAPPEE-NH₂ (SEQ ID NO:2), QETWFQNGWQGKNP (SEQ ID NO:3), KEKWFENEWQGKNP (SEQ ID NO:4), or KEQWFGNRWHEGYR (SEQ ID NO:5). Another CD44-modulating polypeptide can be QIRQQPRDPPTETLELEVSPDPAS (SEQ ID NO:6). Such exemplary peptides can include substitution variants, addition variants, or chemical derivatives thereof including peptidomimetics. Other exemplary CD44-modulating peptides include those set forth in U.S. Pat. Nos. 5,994,309; 6,696,416; and 6,963,587, 8,313,914, 8,697,629, and U.S. Patent Application Publication No. US2009192085.

The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids, and have an activity such as the selective homing activity of the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons (1995), pages 803-861).

The term “prodrug” refers to a compound or polypeptide that is made more active in vivo through the metabolism of a precursor drug. CD44-modulating polypeptides described herein can exist as prodrugs, as described in, for example, Hydrolysis in Drug and Prodrug Metabolism: Chemistry, Biochemistry, and Enzymology (Testa, Bernard, and Mayer, Joachim M. Wiley-VHCA, Zurich, Switzerland 2003). Prodrugs of the polypeptides described herein are structurally modified forms of the peptide that readily undergo chemical changes under physiological conditions to provide the active polypeptide. Additionally, prodrugs can be converted to the active polypeptide by chemical or biochemical methods in an ex vivo environment.

A PD-1 inhibitor refers to a moiety (e.g., compound, nucleic acid, polypeptide, antibody) that decreases, inhibits, blocks, abrogates, or interferes with the activity or expression of PD-1 (e.g., Programmed Cell Death Protein 1; PD-1 (CD279); GI: 145559515), including variants, isoforms, species homologs of human PD-1 (e.g., mouse) and analogs that have at least one common epitope with PD-1. A PD-1 inhibitor includes molecules and macromolecules such as, for example, compounds, nucleic acids, polypeptides, antibodies, peptibodies, diabodies, minibodies, nanobodies, single-chain variable fragments (ScFv), and functional fragments or variants thereof. Thus, a PD-1 inhibitor as used herein refers to any moiety that antagonizes PD-1 activity or expression. PD-1 inhibitor efficacy can be measured, for example, by its inhibitor concentration at 50% (half-maximal inhibitor concentration or IC₅₀). PD-1 inhibitors include exemplary compounds and compositions described herein. A PD-1 antibody refers to a PD-1 inhibitor which is a monoclonal or polyclonal antibody as described herein.

The terms nivolumab, pembrolizumab, pidilizumab, AMP-224, REGN2810, PDR 001, and MEDI0680 are used in accordance with their plain and ordinary meaning as understood in the art.

Provided herein are methods of treating, preventing, managing, or alleviating cancer or symptoms of a cancer in a patient in need thereof. In one aspect is a method of treating a cancer in a patient in need thereof by administering an effective amount of a CD44-modulating polypeptide. In another aspect provided herein is a method of treating cancer in a patient in need thereof by administering an effective amount of a polypeptide comprising the amino acid sequence Ac-KPSSPPEE-NH2 (SEQ ID NO:1, “A6”) or Ac-NASAPPEE-NH2 (SEQ ID NO:2) in combination with an effective amount of an anti-cancer agent; the administration is based on TP mutational status prediction of treatment response. Another aspect provided herein is a method of treating cancer in a patient in need thereof by administering an effective amount of a polypeptide consisting of the amino acid sequence of SEQ ID NO:1 or SEQ ID NO:2 in combination with an effective amount of radiation therapy. Amino acids of polypeptides described herein are numbered in reference from their N to C terminal (or its equivalent). For example, SEQ ID NO:1 can be numbered as follows: Ac-K1P2S3S4P5P6E7E8-NH2.

The present invention features a method of treating a patient with a polypeptide comprising Ac-KPSSPPEE-NH2 (SEQ ID NO:1), an active variant thereof, or a CD44-modulating peptide, wherein the patient has cancer. In some embodiments, the method comprises determining the TP53 mutational status. In some embodiments, TP53 mutational status is determined by 1. obtaining or having obtained a biological sample from the patient and 2. performing or having performed a genotyping assay on the biological sample to determine the TP53 mutational status. In some embodiments, the method comprises administering a polypeptide comprising Ac-KPSSPPEE-NH2 (SEQ ID NO:1), an active variant thereof, or a CD44-modulating peptide, to a patient if the TP53 mutation status predicts a p53 loss of activity. In some embodiments, the polypeptide comprising Ac-KPSSPPEE-NH2 (SEQ ID NO:1), an active variant thereof, or a CD44-modulating peptide is not administered if the TP53 mutational status predicts no tumor p53 expression or TP53 loss of function.

The present invention features a method of determining the effectiveness of a polypeptide comprising Ac-KPSSPPEE-NH2 (SEQ ID NO:1), an active variant thereof, or a CD44-modulating peptide to treat cancer in a patient. In some embodiments, the method comprises determining a TP53 mutational status. In some embodiments, the TP53 mutational status is determined by obtaining or having obtained a biological sample from the patient and performing or having performed a genotyping assay on the biological sample to determine the TP53 mutational status. In some embodiments, the method comprises determining the effectiveness of the polypeptide to treat cancer in the patient based on the TP53 mutational status. In some embodiments, if the TP53 mutational status predicts a p53 loss of activity, the polypeptide is determined to be effective at treating cancer in the patient, and the polypeptide is administered to the patient. In other embodiments, if the TP53 mutational status predicts no tumor p53 expression or TP53 loss of function, the polypeptide is determined to not be effective at treating cancer in the patient, and the polypeptide is not administered to the patient.

As used herein, a “p53 loss of activity” refers to a p53 protein that has a reduction in biological function compared to a wild-type p53 protein. In some embodiments, a loss of p53 activity does not refer to a p53 protein that has lost its biological function.

Without wishing to limit the present invention to any mechanisms or theories it is thought that in order to respond to the SPL-108 treatment, the sample obtained from the patient requires the presence of a functional p53 protein, and therefore, mutations that cause the loss of function of p53 or loss of expression of TP53 are not predicted to respond to treatment with SPL-108.

In some embodiments, if the patient does not have a loss of expression or loss of function, then p53 activity may be restored by SPL-108 treatment leading to a partial response. In other embodiments, If the patient does not have a loss of expression or loss of function, then p53 activity may be restored by SPL-108 treatment leading to a stable disease. In further embodiments, SPL-108 has no effect in patients with loss of expression or loss of function.

In some embodiments, the biological samples obtained from a patient may be genotyped to determine the mutational status of TP53. In other embodiments, the biological sample obtained from a patient may be analyzed by immunohistochemistry analysis to determine if the sample has lost TP53 expression.

In some embodiments, the method further comprises determining TP53 expression. In some embodiments, TP53 expression is determined by obtaining or having obtained a biological sample from the patient and performing or having performed an immunohistochemistry assay on the biological sample to determine TP53 expression.

The present invention also features a composition for use in treating a patient with cancer. In some embodiments, the composition comprises a polypeptide comprising Ac-KPSSPPEE-NH2 (SEQ ID NO:1), an active variant thereof, or a CD44-modulating peptide. In some embodiments, a TP53 mutational status of the patient is used to determine the effectiveness of the composition. In other embodiments, the composition is administered to the cancer patient if the TP53 mutation status predicts a loss of p53 activity. In some embodiments, the composition is not administered if the TP53 mutational status predicts no tumor p53 expression or TP53 loss of function.

Additionally, the present invention further features a method of preventing side effects associated with treating cancer comprising one or more of: gastrointestinal issues, alopecia, allergic reactions, anemia, headache, edema, liver damage, bone marrow suppression, lung infection, osteoporosis, increased risk of blood clots, heart attack, and/or stroke. Non-limiting examples of gastrointestinal issues comprise one or more nausea, vomiting, abdominal pain, acid or sour stomach, ulcers, constipation, diarrhea, black, tarry stools, bloody urine, heartburn, or belching. Non-limiting examples of allergic reactions comprise one or more hives, difficulty breathing, or swelling of the face, lips, tongue, or throat. In some embodiments, the method is for avoiding the one or more side effects commonly associated with treating cancer comprises preventing, reducing the severity, and/or reducing the frequency of one or more gastrointestinal issues, allergic reactions, anemia, headaches, edema, liver damage, bone marrow suppression, lung infection, osteoporosis, risk of blood clots, heart attack, and/or stroke. Non-limiting examples comprise gastrointestinal issues reduced by at least about 20%, allergic reaction reduced by at least about 20%; anemia reduced by at least about 20%, headache reduced by at least about 20%, edema reduced by at least about 20%.

In some embodiments, effectively treating cancer in a subject comprises alleviating clinical symptoms of cancer. Non-limiting examples of clinical symptoms of cancer comprise one or more of: unusual bodily lumps, moles, ulcerations, fluid accumulation, and fluid discharge, pain, inflammation.

In some embodiments, the biological sample is selected from a group consisting of tissue, blood, and cells, saliva, and/or cerebrospinal fluid (CSF). In some embodiments, tissue may comprise fresh tissue, frozen tissue, and/or formalin-fixed, paraffin-embedded (FFPE).

In preferred embodiments, determining TP53 mutational status comprise using standard laboratory techniques comprising polymerase chain reaction (PCR), real-time PCR, transcription-mediated amplification (TMA) and reverse transcription PCR (RT-PCR), sequencing, microarray sequencing, next-generation sequencing.

In some embodiments, the TP53 mutation may be in the oligomerization domain. In other embodiments, the TP53 mutation may cause a non-functional protein. In some embodiments, the TP53 mutation may cause a truncated protein. In other embodiments, the TP53 mutation may be a nonsense mutation. In some embodiments, the TP53 mutation may be a frameshift mutation. In other embodiments, the TP53 mutation may be a point mutation. In some embodiments, a point mutation may comprise an insertion or a deletion mutation. In some embodiments, the TP53 mutational status comprises a mutation selected from the group consisting of: R342* (* mutation in oligomerization domain), D259V (non-functional protein), R175H, R273C, R213* (* truncated/nonsense), G105fs* (* truncated/frameshift), E286Q, C238W, and wildtype.

In some embodiments, the TP53 mutation status that predicts a partial response comprises mutations selected from the group consisting of R342*, R273C, and C238W. In other embodiments, the TP53 mutation status that predicts a stable disease comprises mutations selected from the group consisting of R175H, E286Q, and no mutation (wildtype (WT)). In further embodiments, the TP53 mutation status that predicts a progressive disease comprises mutations selected from the group consisting of D259V, R213*, and G105fs.

In some embodiments, patients with progressive disease are predicted not to respond to SPL-108 treatment. In other embodiments, patients with a partial response or stable disease are predicted to respond to SPL-108 treatment.

In some embodiments, mutations that predict a progressive disease should not respond to SPL-108 treatment. In other embodiments, mutations that predict a partial response or stable disease should respond to SPL-108 treatment. In some embodiments, mutations that predict a progressive disease will not respond to SPL-108 treatment. In other embodiments, mutations that predict a partial response or stable disease will respond to SPL-108 treatment.

In some embodiments, if the patient does not have a loss of expression of p53 or TP53 loss of function, then p53 activity may be restored by SPL-108 treatment leading to a partial response. In other embodiments, if the patient does not have a loss of expression of p53 or TP53 loss of function, then p53 activity may be restored by SPL-108 treatment leading to stable disease. In further embodiments, SPL-108 has no effect in patients with loss of expression or loss of function.

In some embodiments, the response to SPL-108 treatment is characterized by a lack of function of TP53. In some embodiments, TP53 mutations that cause a loss in p53 expression or TP53 loss of function are not expected to respond to SPL-108. In other embodiments patients without tumor p53 expression or TP53 loss of function are not expected to respond to SPL-108.

In preferred embodiments, these methods comprise first determining TP53 mutational status in a biological sample (e.g., tissue, cells, blood, saliva, cerebrospinal fluid CSF) obtained from the patient with cancer. The patient is then screened and/or selected to be eligible to receive a treatment comprising a polypeptide, for example, the polypeptide is Ac-KPSSPPEE-NH2 (SEQ ID NO:1), an active variant thereof, or a CD44-modulating peptide based on the determined TP53 mutational status in a biological sample obtained from the patient. Then during a first period, the treatment comprising SEQ ID NO:1, an active variant thereof, or a CD44-modulating peptide, is administered to the screened or selected patient afflicted with cancer at an effective daily dose over a period of time sufficient to effectively treat the cancer. Without wishing to limit the present invention to any particular theory or mechanism, the TP53 mutational status predicts the response of the patient to SPL-108 an active variant thereof or a CD44-modulating peptide treatment.

In some embodiments, the first period comprises a period of time sufficient to effectively treat the cancer including alleviating one or more clinical symptoms of cancer. Non-limiting examples of the first time period comprise at least 1 day, at least 2 days, at least 7 days, at least 14 days, at least 21 days, at least 42 days, at least 60 days, at least 3 months, at least 6 months, at least 1 year, or until the one or more clinical symptoms of cancer are no longer alleviated in the subject. In some embodiments, the methods further comprise a second period.

In some embodiments, the CD44-modulating polypeptide is a polypeptide comprising SEQ ID NO:1 or SEQ ID NO:2. The polypeptide of SEQ ID NO:1 can be a capped 8-amino acid peptide. The sequence of the polypeptide of SEQ ID NO:1 corresponds to amino acid residues 136-143 of the connecting peptide domain of the human urokinase plasminogen activator (uPA). The connecting peptide domain is located between the N-terminal growth factor domain and the C-terminal catalytic domain of uPA. The N-terminal growth factor domain of uPA binds to the uPA receptor (uPAR) to initiate the uPA/uPAR cascade. The binding of uPA to uPAR can initiate a cascade of events leading to proteolysis, degradation of the extracellular matrix (ECM), cell migration, cell invasion, metastasis, and angiogenesis. Such events can promote cell death, including cell death of cancer cells in solid tumors and hematological cancers. The uPA system has been shown to play a role in the growth and spread of solid tumors. Levels of uPA and uPAR can correlate with clinical outcomes in a variety of malignancies. In certain instances, the upregulation of the uPA system can be associated with a poor prognosis. The inhibition of the uPA system can block critical processes (e.g., cell migration, invasion, and angiogenesis) useful for a broad range of proliferative diseases.

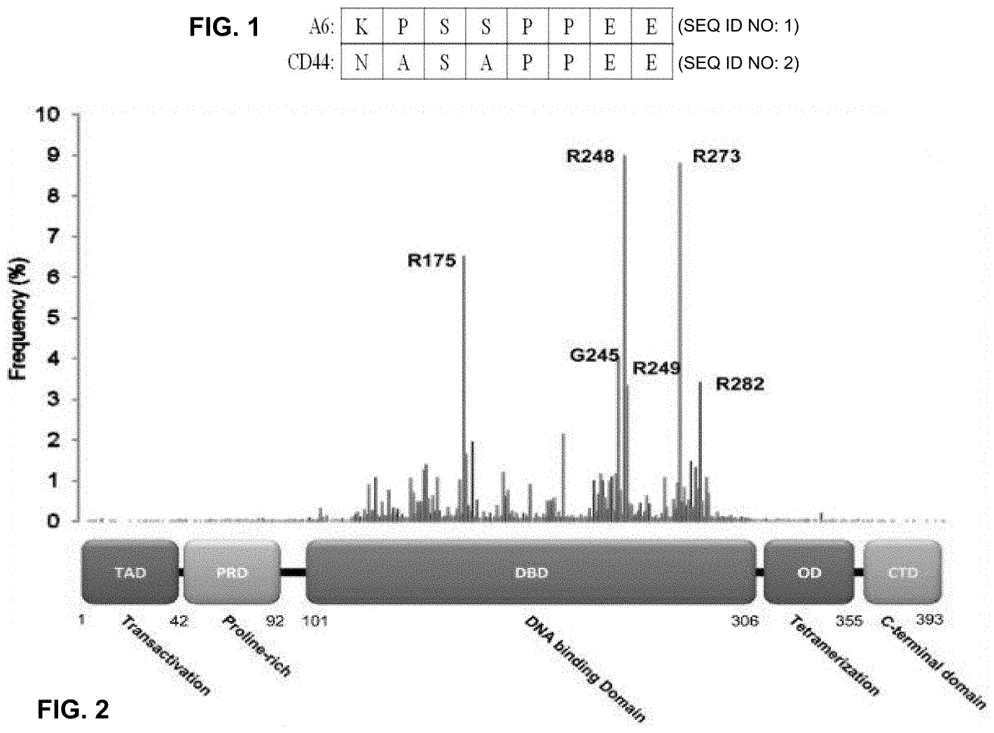

The polypeptide of SEQ ID NO:1 shares sequence homology with a portion of the Link-Domain of CD44 (CD44 amino acid residues 120-NASAPPEE-127) (FIG. 1). The CD44 gene is encoded by 20 exons in the mouse and 19 exons in humans. There are 5 constant exons expressed at the 5′ end, and 10 variant exons (mouse) or 9 variant exons (human) can be alternatively spliced within CD44 at an insertion site after the fifth constitutive exon, followed by the remaining constant exons at the 3′ end. The smallest isoform of CD44 (CD44s) contains no variant exons. The largest isoform of CD44 (CD44v1-10) contains all of the variant exons. SEQ ID NO:1 can be found in nearly all CD44 isoforms, in part, because it is located within the first 5 non-variable exons of the isoform. SEQ ID NO:1 is located at the CD44 splice junction of exons 3 and 4.

The SEQ ID NO:1 can include a substitution of K to A; P to A; S to A; or E to A. In some embodiments, the sequence of SEQ ID NO:1 can be modified such that the CD44-modulating polypeptide includes a mutation of K1 to A so long as the polypeptide retains activity about equal to or greater than the polypeptide of SEQ ID NO:1. In another embodiment, SEQ ID NO: 1 can be modified to include mutation of P2, P5, P6, or a combination thereof to A so long as the polypeptide retains activity about equal to or greater than the polypeptide of SEQ ID NO:1. In certain embodiments, P2 can be mutated to A. In certain embodiments, P5 can be mutated to A. In certain embodiments, P6 can be mutated to A. In another embodiment, S3, S4, or S3 and S4 can be mutated to A so long as the polypeptide retains activity about equal to or greater than the polypeptide of SEQ ID NO:1. In another embodiment, E7, E8, or E7 and E8 can be mutated to A so long as the polypeptide retains activity about equal to or greater than the polypeptide of SEQ ID NO:1. It is to be understood that the above mutations can be combined in any manner to modify the polypeptide of SEQ ID NO:1 so long as the polypeptide retains activity about equal to or greater than the polypeptide of SEQ ID NO:1.

SEQ ID NO:1 can include at least one glycosylation site. The glycosylation site can be an O-linked glycan on S3, S4, or S3 and S4 of SEQ ID NO:1. In other instances, the glycosylation site can be present in any one Ser or Thr residue of SEQ ID NOs:1-6.

The following sequences are non-limiting examples of mutation in the polypeptide

| SEQUENCE: | SEQ ID NO: |
|---|---|
| Ac-KPSSPPEE-NH2 | 1 |
| AC-NASAPPEE-NH2 | 2 |
| QETWFQNGWQGKNP | 3 |
| KEKWFENEWQGKNP | 4 |
| KEQWFGNRWHEGYR | 5 |
| QIRQQPRDPPTETLELEVSPDPAS | 6 |
| Ac-APSSPPEE-NH₂ | 7 |
| Ac-KASSPPEE-NH₂ | 8 |
| Ac-KPSSAPEE-NH₂ | 9 |
| Ac-KPSSPAEE-NH₂ | 10 |
| AC-KASSPAEE-NH2 | 11 |
| AC-KPSSAAEE-NH2 | 12 |
| Ac-KPASPPEE-NH₂ | 13 |
| Ac-KPSAPPEE-NH₂ | 14 |
| Ac-KPAAPPEE-NH₂ | 15 |
| Ac-KPSSPPAE-NH₂ | 16 |
| Ac-KPSSPPEA-NH₂ | 17 |
| Ac-KPSSPPAA-NH₂ | 18 |
| Ac-APSSPPAE-NH₂ | 19 |
| Ac-KPSAAPEE-NH₂ | 20 |

The CD44-modulating polypeptides described herein used in combination with one or more anti-cancer agents described herein, radiation therapy as described herein, or a combination thereof, are useful for treating, preventing, and/or managing cancer of the skin tissues, organs, blood, and vessels, including, but not limited to, cancers of the bladder, bone, blood, brain, breast, cervix, chest, colon, endometrium, esophagus, eye, head and neck, kidney, liver, lymph nodes, lung, mouth, ovaries, pancreas, peritoneal, prostate, rectum, stomach, testis, throat, and uterus. In one embodiment, cancers include, but are not limited to, advanced malignancy, amyloidosis, neuroblastoma, meningioma, hemangiopericytoma, multiple brain metastasis, glioblastoma multiforme, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, follicular lymphoma, low grade follicular lymphoma, acute myelogenous leukemia, acute lymphocytic leukemia, chronic myelogenous leukemia, chronic lymphocytic leukemia, malignant melanoma, malignant mesothelioma, malignant pleural effusion mesothelioma syndrome, peritoneal carcinoma, primary peritoneal cancer, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. The CD44-modulating polypeptide can be a polypeptide of SEQ ID NO:1 or SEQ ID NO:2.

In some embodiments, the cancer can be a solid tumor. The cancer can be a solid tumor selected from the group consisting of squamous cell carcinoma, nonsquamous cell carcinoma, non-small cell lung cancer (NSCLC), small cell lung cancer, renal cell carcinoma, ovarian cancer, breast cancer, prostate cancer, head and neck cancer, endometrial cancer, primary peritoneal cancers, glioblastoma, colorectal cancer, or pancreatic cancer. In one embodiment, a CD44-modulating polypeptide can be administered in combination with an anti-cancer agent described herein, radiation therapy described herein, or a combination thereof, for treating breast cancer, ovarian cancer, or endometrial cancer. In another embodiment, a CD44-modulating polypeptide can be administered in combination with an anti-cancer agent described herein, radiation therapy described herein, or a combination thereof, for treating colorectal cancer, peritoneal cancer, or renal cell carcinoma. In another embodiment, a CD44-modulating polypeptide can be administered to treat NSCLC or small cell lung cancer.

In some embodiments, the cancer can be a hematological cancer. Hematological cancer can be leukemia, such as for example Acute Myeloid Leukemia (AML). In one embodiment, a CD44-modulating polypeptide can be administered in combination with an anti-cancer agent described herein for treating AML

In some aspects, the cancer can be resistant to at least one anti-cancer agent or radiation therapy. The cancer may be a resistant or refractory cancer. In some embodiments, the cancer is resistant or refractory to treatment with chemo-, immuno-, gene, and/or radiation therapy.

Thus, the present invention provides methods for treating a resistant or refractory cancer by administering a CD44-modulating polypeptide as described herein in combination with an anti-cancer agent described herein. In another embodiment, methods for treating a resistant or refractory cancer comprise administering a CD44-modulating polypeptide provided herein in combination with radiation therapy described herein. In other embodiments, the methods for treating a resistant or refractory cancer comprise administering a CD44-modulating polypeptide provided herein in combination with an anti-cancer agent and radiation therapy as described herein. It is well known in the art that many cancers are resistant or refractory to many anti-cancer agents or radiation therapy(ies) or over the course of treatment, become resistant or refractory to treatment. The methods described herein can restore the activity of anti-cancer agents having reduced or eliminated activity against one or more cancers and permit additional treatment options for cancer patients. In another embodiment, the methods described herein can restore the activity of radiation therapies described herein having reduced or eliminated activity against one or more cancers and permit additional treatment options for cancer patients.

In other embodiments, the cancer can optionally be resistant or refractory to a plurality of anti-cancer agents (e.g. two or more anti-cancer agents) and/or a plurality of radiation therapies. In one example the cancer can also be resistant, refractory, or non-responsive to treatment with a CD44-modulating polypeptide described herein. In one embodiment of methods of treating described herein, a patient can be administered a combination of a CD44-modulating polypeptide described herein and an anti-cancer agent where the cancer treated is resistant, refractory, or non-responsive to one of or both the CD44-modulating polypeptide and the anti-cancer agent. In one example, the cancer can be resistant, refractory, or non-responsive to treatment with the anti-cancer agent. The administration of the combination of the CD44-modulating polypeptide and anti-cancer agent(s) surprisingly can restore or enhance the activity of the anti-cancer agent against the refractory, resistant, or non-responsive cancer. Administration of the combination of the CD44-modulating polypeptide and anti-cancer agent(s) surprisingly can restore or enhance the activity of the CD44-modulating polypeptide against the refractory, resistant, or non-responsive cancer. The administration of the combination of the CD44-modulating polypeptide and anti-cancer agent(s) surprisingly can restore or enhance the activity of the CD44-modulating polypeptide and the anti-cancer agent against the refractory, resistant, or non-responsive cancer.

In some embodiments, the methods described herein further comprise administering a standard-of-care therapy, an anti-cancer agent, and/or radiation therapy. In some embodiments, standard-of-care therapy comprises analgesics, anti-inflammatory agents, anti-proliferative agents, corticosteroids, hormone therapy, immunotherapy, gene therapy, radiotherapy, and/or biological response modifiers administered to the subject suffering from cancer. In some embodiments, the anti-cancer agent is selected from the group consisting of avastin, paclitaxel, paclitaxel analogues, docetaxel, cabazitaxel, doxorubicin, a checkpoint inhibitor, a PARP inhibitor, methotrexate, cisplatin, oxaliplatin, and carboplatin. In other embodiments, the PARP inhibitor is talazoparib, olaparib, or rucaparib.

The anti-cancer agent can be paclitaxel, a paclitaxel analogue, docetaxel, cabazitaxel, doxorubicin, a checkpoint inhibitor, cisplatin, oxaliplatin, carboplatin, methotrexate, or a PARP inhibitor. In one embodiment, the anti-cancer agent can be methotrexate. In certain embodiments, the anti-cancer agent can be paclitaxel, a paclitaxel analogue, docetaxel, cabazitaxel, doxorubicin, a checkpoint inhibitor, cisplatin, oxaliplatin, carboplatin, methotrexate, or a PARP inhibitor which can be administered in combination with a polypeptide of SEQ ID NO:1 or a polypeptide of SEQ ID NO:2. It is to be understood that the embodiments described herein can further include administering one or more radiation therapies as described herein

The anti-cancer agent can be a PARP inhibitor. The PARP inhibitor can inhibit one or both PARP1 and PARP2. The PARP inhibitor can be talazoparib (BMN 673), olaparib, rucaparib, iniparib, or veliparib. The cancer treated can be resistant, refractory, or non-responsive to PARP inhibitor treatment. The combination of a PARP inhibitor and CD44-modulating polypeptide (e.g. SEQ ID NO:1 or SEQ ID NO:2) can restore or enhance the anti-cancer activity of the PARP inhibitor against the cancer.

The energy source used for the radiation therapy may be selected from X-rays or gamma rays, which are both forms of electromagnetic radiation. X-rays can be created by machines called linear accelerators. Depending on the amount of energy the x-rays have, they can be used to destroy cancer cells on the surface of the body (in the case of lower energy rays), or deeper into tissues and organs (in the case of higher energy rays). Compared with other types of radiation, x-rays can deliver radiation to a relatively large area. Gamma rays can be produced by isotopes of certain elements, such as iridium and cobalt 60, which release radiation energy as they decay. Each element decays at a specific rate and each gives off a different amount of energy, which affects how deeply it can penetrate into the body. Gamma rays produced by the decay of cobalt 60 are used in the treatment referred to as the “gamma knife.”

The energy source for the radiation therapy may be selected from particle beams, which typically use fast-moving subatomic particles instead of photons. This type of radiation may be referred to as particle beam radiation therapy or particulate radiation. Particle beams may be created by linear accelerators, synchrotrons, betatrons, and cyclotrons, which produce and accelerate the particles. Particle beam therapy may use electrons, which can be produced by an x-ray tube; neutrons, which can be produced by radioactive elements and special equipment; heavy ions such as protons, carbon ions, and helium; and/or pi-mesons, also called pions, which are small, negatively charged particles produced by an accelerator and a system of magnets. Unlike x-rays and gamma rays, some particle beams, depending on the energy, can penetrate only a short distance into tissue. Therefore, they may be used to treat cancers located on the surface of or just below the skin.

In some embodiments, radioactive isotopes are the source of ionizing radiation for radiation therapy. Non-limiting examples of radioactive isotopes include radioactive isotopes of iodine (e.g. iodine 125 or iodine 131), strontium, phosphorous, palladium, cesium, iridium, phosphate, samarium, yttrium, or cobalt (e.g. cobalt 60). In particular, iodine 125 (t 1/2=60.1 days), palladium 103 (t 1/2=17 days), cesium 137, strontium 89 (t 1/2=50.5 days), samarium 153 (t 1/2=46.3 hours), and iridium 192, can advantageously be used. Radiation may be delivered directly to the cancer through the use of radiolabeled antibodies, also referred to as radioimmunotherapy. This approach can reduce or minimize the risk of radiation damage to healthy cells. In certain embodiments, the radioimmunotherapy treatments are selected from ibritumomab tiuxetan (Zevalin®) and tositumomab, and iodine 131 tositumomab (Bexxar®). Radioimmunotherapy may be used in the treatment of advanced adult non-Hodgkin lymphoma (NHL). In certain embodiments, immunotherapy is used in the treatment of cancers including leukemia, NHL, colorectal cancer, and cancers of the liver, lung, brain, prostate, thyroid, breast, ovary, and pancreas.

In some embodiments, the radiation therapy is selected from the group consisting of X-Rays, gamma-Rays, UV-Rays, a particle beam, and decay of a radioactive isotope. In other embodiments, the particle beam is selected from the group consisting of an electron beam, a neutron beam, a proton beam, a carbon ion beam, and a pion beam. In some embodiments, the radioactive isotope is a radioactive isotope of iodine, strontium, phosphorus, palladium, cesium, iridium, phosphate, samarium, yttrium, or cobalt. In some embodiments, the radioactive isotope is iodine 125 or iodine 131, In other embodiments, the radioactive isotope is cobalt 60.

Combinations described herein that include a CD44-modulating polypeptide described herein and an anti-cancer described herein can be provided as a pharmaceutical composition suitable for administration via any route to a patient described herein including but not limited to: oral, mucosal (e.g., nasal, inhalation, pulmonary, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient. The CD44-modulating polypeptide can be a polypeptide of SEQ ID NO:1 or SEQ ID NO:2. In particular embodiments, a CD44-modulating polypeptide described herein can be formulated as a component of a pharmaceutical composition suitable for transdermal delivery.

Exemplary of dosage forms include transdermal systems, tablets; caplets; capsules (e.g., gelatin capsules); cachets; lozenges; suppositories; powders; gels; liquid dosage forms suitable for parenteral administration to a patient; and sterile solids (e.g., crystalline or amorphous solids) that can be reconstituted to provide liquid dosage forms suitable for parenteral administration to a patient. In certain embodiments, the CD44-modulating polypeptide described herein can be formulated for parenteral or oral administration. In certain embodiments, the anti-cancer agent described herein can be formulated for transdermal, parenteral, nasal, or oral administration. In certain embodiments, the CD44-modulating polypeptide can be formulated for parenteral or transdermal dosing. In another embodiment, the CD44-modulating polypeptide can be formulated for nasal or parenteral administration

In one example, the CD44-modulating polypeptide can be formulated for transdermal administration. Transdermal administration includes extended/sustained release transdermal devices (e.g., the release of the polypeptide for a period of more than about 1 hour or 1 or more days) and immediate release transdermal devices (e.g., the release of the polypeptide over a period of time of less than about 1 hour). In certain instances, the CD44-modulating polypeptide can be administered using an immediate release transdermal device that releases the CD44-modulating polypeptide over the course of about 0.5, 1, 2, 3, 4, 5, 6, 7, 8, 9, 10, 11, 12, 13, 14, 15, 20, 25, 30, 45, or 60 minutes. For example, the immediate release transdermal device can release the CD44-modulating polypeptide over a period of time of less than about 60 min; less than about 30 min; less than about 20 min, less than about 15 min; less than about 10 min; less than about 5 min; less than about 3 min; less than about 2 min; less than about 1 min; or less than about 0.5 min. In one embodiment, transdermal delivery of a CD44-modulating polypeptide described herein reduces metabolic degradation of the CD44-modulating polypeptide.

In certain instances, transdermal administration can be advantageous for promoting patient compliance because transdermal administration is noninvasive and the administration can be completed in less than a time period set forth above. In such embodiments, the amount of the CD44-modulating polypeptide administered to a patient can be increased by transdermal administration. Patient compliance is often an impetus for failed therapies because fear, pain, and the process of administration can deter patient compliance. Transdermal administration of CD44-modulating polypeptides described herein can increase patient compliance and increase treatment success.

In some embodiments, the administration of SEQ ID NO:1 is parenteral administration comprising subcutaneous administration, intradermal administration, and/or intranasal administration. In some embodiments, the dose of SEQ ID NO:1 ranges from 5 mg to 50 mg, administered twice daily. In some embodiments, the dose of SEQ ID NO:1 ranges from 50 mg to 100 mg, administered twice daily. In some embodiments, the dose of SEQ ID NO:1 ranges from 100 mg to 150 mg, administered twice daily. In some embodiments, the method further comprises, during a second period, giving the subject a drug holiday from administering SEQ ID NO:1. In some embodiments, the drug holiday comprises withholding SEQ ID NO:1 administration for a period of time. In some embodiments, the drug holiday comprises withholding SEQ ID NO:1 administration for a period of time, wherein one or more clinical symptoms of cancer continue to be alleviated. In some embodiments, the method further comprises, during a second period, reducing the daily dose of SEQ ID NO:1. In some embodiments, the method further comprises, during the second period, reducing the daily dose of SEQ ID NO:1 to 5 mg from 10 mg per day. In some embodiments, the method further comprises, during a second period, increasing the daily dose of SEQ ID NO:1. In some embodiments, the method further comprises during the second period, increasing the daily dose of SEQ ID NO:1 from 300 mg to 600 mg per day. In some embodiments, the second period comprises at least 1 day, at least 2 days, at least 7 days, at least 14 days, at least 42 days, at least 60 days, or until one or more clinical symptoms of cancer are no longer alleviated in the patient.

In other embodiments, the Ac-KPSSPPEE-NH2 (SEQ ID NO:1), an active variant thereof, or a CD44-modulating peptide is administered continuously and without a rest period

A CD44-modulating polypeptide described herein can be administered in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 900 mg, 1000 mg, 1100 mg, 1200 mg, 1300 mg, 1400 mg, or 1500 mg. A CD44-modulating polypeptide described herein can be administered in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, or 450 mg. A CD44-modulating polypeptide described herein can be administered in an amount of about: 100 mg, 125 mg, 150 mg, 175 mg, 200 mg, 250 mg, 300 mg, 350 mg, or 400 mg

A CD44-modulating polypeptide can be administered in an amount from about: 1 mg to about 500 mg: 50 mg to about 500 mg; 100 mg to about 500 mg; 150 mg to about 500 mg; 250 mg to about 500 mg; or about 300 mg to about 500 mg. A CD44-modulating polypeptide can be administered in an amount from about: 100 mg to about 200 mg; 100 mg to about 250 mg; 100 mg to about 300 mg; or 100 mg to about 400 mg.

A CD44-modulating polypeptide can be administered in an amount from about: 1 mg to about 1500 mg: 50 mg to about 1500 mg; 100 mg to about 1500 mg; 250 mg to about 1500 mg; 500 mg to about 1500 mg; or about 1000 mg to about 1500 mg. A CD44-modulating polypeptide can be administered in an amount from about: 100 mg to about 2000 mg; 100 mg to about 3000 mg; 100 mg to about 4000 mg; 100 mg to about 5000 mg; 100 mg to about 6000 mg; 100 mg to about 7000 mg; 100 mg to about 8000 mg; 100 mg to about 9000 mg; or 100 mg to about 10,000 mg. A CD44-modulating polypeptide can be administered in an amount from about: 500 mg to about 2000 mg; 500 mg to about 1750 mg; 1000 mg to about 2000 mg; 1000 mg to about 1500 mg; 1200 mg to about 1800 mg; or about 1300 mg to about 1500 mg. A CD44-modulating polypeptide can be administered in an amount from about: 1000 mg to 10000 mg; 2000 mg to 10000 mg; 1000 mg to 7500 mg; 1000 mg to 5000 mg; 750 mg to about 4000 mg; or 2000 mg to 5000 mg.

A CD44-modulating polypeptide described herein can be administered as described herein in an amount of about: 25 mg/day to about 1500 mg/day; 25 mg/day to about 1200 mg/day; 25 mg/day to about 1000 mg/day; 25 mg/day to about 750 mg/day; 25 mg/day to about 500 mg/day; 25 mg/day to about 300 mg/day; 25 mg/day to about 250 mg/day; 25 mg/day to about 150 mg/day; 50 mg/day to about 1000 mg/day; 50 mg/day to about 750 mg/day; 50 mg/day to about 500 mg/day; 50 mg/day to about 300 mg/day; 50 mg/day to about 250 mg/day; 50 mg/day to about 150 mg/day; 100 mg/day to about 500 mg/day; 100 mg/day to about 300 mg/day; or about 150 mg/day to about 300 mg/day.

In certain instances it can be useful to administer the CD44-modulating polypeptide as an amount relative to the weight of the patient. A polypeptide of SEQ ID NO:1 or SEQ ID NO:2 can be administered as an amount relative to the weight of the patient. A polypeptide of SEQ ID NO:1 or SEQ ID NO:2 can be administered in an amount of about: 0.5 mg/kg, 1 mg/kg, 2 mg/kg, 3 mg/kg, 5 mg/kg, 6 mg/kg, 7 mg/kg, 8 mg/kg, 9 mg/kg, 10 mg/kg, 15 mg/kg, or 20 mg/kg. In certain instances the polypeptide of SEQ ID NO:1 or SEQ ID NO:2 can be administered in an amount of about 0.5 mg/kg to about 20 mg/kg; 0.5 mg/kg to about 10 mg/kg; 0.5 mg/kg to about 7.5 mg/kg; 0.5 mg/kg to about 5 mg/kg; or about 0.5 mg/kg to about 1 mg/kg.

CD44-modulating polypeptides can be administered in a treatment regimen that includes administration of the polypeptide in any number of days, weeks, or months and over any period of time, typically until disease lapse, unacceptable toxicity, patient intolerance, or onset of disease symptoms (e.g. relapse or loss of efficacy). CD44-modulating polypeptides described herein and useful in the methods of treating cancers described herein can be administered at any frequency as described herein such as, for example, once a day (QD), every other day (Q2D), twice daily (BID), once a week (QW), twice weekly (BIW), three times a week (TIW), every other week (Q2W), every three weeks (Q3W), or monthly (QM). In one example a CD44-modulating polypeptide described herein can be administered QD for at least 5, 10, 15, 21, 28, 30, 31, 45, 60, 90, 120, 180, or 200 days. In certain instances, the CD44-modulating polypeptide can be administered QD for at least 30 or 60 days. In another instance, the CD44-modulating polypeptide can be administered QD for at least 90 or 180 days. In one example the CD44-modulating polypeptide can be administered QD until the progression of the disease or toxicity development.

In another example, a CD44-modulating polypeptide can be administered as maintenance therapy before, during, or after treatment with an anti-cancer agent described herein, radiation therapy described herein, or a combination thereof. Maintenance therapy refers to long-term (e.g., 6 months, or 1, 2, 3, 4, 5, 6 or more years) treatment following a treatment regimen for cancer, such as those described herein, that is intended to keep the cancer in remission. Maintenance therapy can be administered indefinitely following a treatment regimen described herein. In certain instances, CD44-modulating polypeptides have little or no toxicity to the patient and continual administration, even after ending a treatment with an anti-cancer agent described herein, radiation therapy described herein, or a combination thereof. In one example, a CD44-modulating polypeptide can be administered as a maintenance therapy after the course of a regimen described herein is completed.

In one embodiment, a CD44-modulating polypeptide can be administered QD at an amount of about 100 mg to 400 mg. In one embodiment, a CD44-modulating polypeptide can be administered QD at an amount of about 1000 mg to 2000 mg. In one embodiment, a CD44-modulating polypeptide can be administered QD at an amount of about 150 mg to 300 mg. In one embodiment, a CD44-modulating polypeptide can be administered QD at an amount of about 100 mg to about 200 mg or about 250 mg to about 350 mg.

In one example, a CD44-modulating polypeptide can be administered BID at an amount of about 100 mg to 400 mg. In one embodiment, a CD44-modulating polypeptide can be administered BID at an amount of about 1000 mg to 2000 mg. In one embodiment, a CD44-modulating polypeptide can be administered BID at an amount of about 150 mg to 300 mg. In one embodiment, a CD44-modulating polypeptide can be administered BID at an amount of about 100 mg to about 200 mg or about 250 mg to about 350 mg. In one embodiment a CD44-modulating polypeptide can be administered BID at an amount of about 150 mg.

In another example, a CD44-modulating polypeptide can be administered multiple times a day such that the total amount administered in one day (e.g., about 24 hours) can be about 100 mg to about 400 mg. A CD44-modulating polypeptide can be administered multiple times such that the total amount administered in one day can be about 100 mg, 150 mg, 200 mg, 250 mg, 300 mg, or about 350 mg.

In another example, a CD44-modulating polypeptide described herein can be administered Q2D. The duration of administration can be the same as described above for QD administration. For example, Q2D administration can be performed for at least 30, 60, 90, 120, or 180 days. In one embodiment, the amount of CD44-modulating polypeptide administered Q2D can be equivalent to an amount administered QD. In another embodiment, the amount of CD44-modulating polypeptide administered Q2D can be greater than the amount administered QD.

In another example, a CD44-modulating polypeptide can be administered once weekly (QW). Once weekly administration can be performed for at least 1, 2, 3, 4, 5, 6, 7, 8, 9, 10, 11, 12, 16, 20, or 24 weeks.

QD or BID administration of CD44-modulating polypeptides can be performed in a cyclic regimen that includes administration as described above, e.g. 14, 21, or 28 days, and the cycle repeated continually. In one example, QD or BID administration of a CD44-modulating polypeptide can be continuous without any rest or off period of administration. In another example, administration includes a rest period or off period can be included between each cycle. A rest or off period can be about 1 to 7 days.

### EXAMPLE

The following is a non-limiting example of the present invention. It is to be understood that said example is not intended to limit the present invention in any way. Equivalents or substitutes are within the scope of the present invention.

### Example 1: Safety and Efficacy Study of Daily SPL-108 Injections in Conjunction with Paclitaxel in Women with Ovarian Cancer; SPL-006

SPL-006 is entitled, An Open-label Phase 1 Trial of the Safety and Efficacy of Daily Subcutaneous SPL-108 Injections When Used In Combination With Paclitaxel In Patients With Platinum-resistant, CD44+, Advanced Ovarian Epithelial Cancer. SPL-006 is an open-label 2-arm clinical trial that is currently ongoing to examine the safety and efficacy of daily SPL-108 injections in conjunction with paclitaxel in women with ovarian cancer; SPL-006. Up to 18 women with ovarian cancer are administered up to two injections of SPL-108 daily in combination with weekly doses of paclitaxel. The women are monitored for safety and efficacy for up to 6 months until disease progression or unacceptable toxicity.

Primary outcome includes incidence of Adverse Events (Safety)Time Frame: Until dose-limiting toxicity, disease progression or 6 months] Includes adverse events, laboratory data, physical examination findings, vital signs, weight, and ECOG Performance Status.

Secondary Endpoints include: Response to Investigational Product [time frame: Until dose-limiting toxicity, disease progression or 6 months]; with the objective response (OR) being 1. through imaging assessment 2. through changes in CA 125 levels; 3. assessed through changes in Quality of Life (FACT-O Questionnaire).

Eligible subjects are screened and enrolled sequentially into 1 of 2 Arms:

Arm I: N=6 to 12 subjects, Safety Phase.

Cohort 1 SPL-108×1 injection daily+80 mg/m2 PTX weekly on Days 1, 8, and 15 in 28 day cycles. Cohort 2 SPL-108 BID+80 mg/m2 PTX weekly on Days 1, 8, and 15 in 28 day cycles.

For Arm I, at least 1 week will elapse between Dose 1 for each subject.

In Cohort 1, 3 subjects will be enrolled: if 1 dose-limiting toxicity (DLT) occurs, 3 subjects will be added at this dose level; if 0/3 or 1/6 DLTs occur, Cohort 2 will initiate enrolling subjects; if 2 or more DLTs occur at this dose level, subjects will not be enrolled in Cohort 2 and the trial will be terminated. In Cohort 2, 3 subjects will be enrolled: if 1 DLT occurs, 3 subjects will be added at this dose level; if 0/3 or 1/6 DLTs occur, Arm II will initiate enrolling subjects at the SPL-108 BID dose; if 2 or more DLTs occur, Arm II will initiate enrolling subjects at the SPL-108 dose one time each day.

Arm II: N=up to 12, Exploratory Expansion Phase

Cohort 3: SPL-108 daily dose (to be determined in Arm I)+80 mg/m2 PTX weekly on Days 1, 8, and 15 in 28-day cycles.

A Safety Committee meets at the end of each Arm I Cohort as well as periodically to review safety data and make recommendations for trial progression. The primary efficacy outcome, Response Evaluation Criteria in Solid Tumors Committee (RECIST 1.1) criteria, is assessed on Day 15 of Cycles 2, 4, and 6.

TABLE 1
SPL-008 Study Summary

| | |
|---|---|
| Actual Enrollment: | 14 participants |
| Allocation: | Non-Randomized |
| Intervention Model: | Sequential Assignment |
| Intervention Model | Arm I: N = 6 to 12, Safety Phase, 3 + 3 design with |
| Description: | 2 cohorts. |
| | Cohort 1 SPL-108 150 mg daily + 80 mg/m2 PTX |
| | weekly on Days 1, 8, and 15 in 28 day cycles |
| | Cohort 2 SPL-108 300 mg daily (150 mg q12 |
| | hours) + 80 mg/m2 PTX weekly on Days 1, 8, and |
| | 15 in 28 day cycles |
| | Arm II: N = up to 12, Exploratory Expansion Phase |
| | Cohort 3: SPL-108 daily dose (to be determined in |
| | Arm I) + 80 mg/m2 PTX weekly on Days 1, 8, and |
| | 15 in 28-day cycles. |
| Masking: | None (Open Label) |
| Primary Purpose: | Treatment |

### Preliminary Results

Table 2 shows preliminary results of patient response to SPL-108 based on TP53 mutational status in the SPL-108 study. Of the 11 patients, three (3) patients had progressive disease (PD), four (4) patients had stable disease, and four (4) had a partial response (PR) response to SPL-108 treatment. In the patients with a lack of response, the lack of response may be characterized by a lack of function of TP53. Of the 3 that did not have a response, 2 had TP53 truncations and the third had a mutation resulting in a non-functional protein, demonstrating the importance of lack of function alterations.

TABLE 2
SPL-006 Response Data Summary and p53 Mutational Status.

| | Best | | | |
|---|---|---|---|---|
| | Response | | | IARC |
| Patient | FINAL | p53 | p53 | TP53 |
| # | SCORE | Mutation | Domain | Database |
| 1 | PR | R342* | Oligomerization | |
| | | | Domain | |
| 2 | PD | D259V | L3: DNA-Prot | Non-Functional |
| | | | Interaction | Protein |
| 3 | SD | R175H | L2: folding/ | Missense |
| | | | stabilization | |
| 4 | PR | R273C | DNA Binding Domain | Missense |
| 5 | PD | R213* | DNA Binding Domain | Truncated/ |
| | | | | Nonsense |
| 6 | PD | G105fs* | DNA Binding Domain | Truncated/ |
| | | | | Frame Shift |
| 7 | SD | E286Q | DNA Binding Domain | Missense |
| 8 | PR | Unknown | | |
| 9 | SD | Unknown | | |
| 10 | SD | WT | Wild type | |
| 11 | PR | C238W | L3: DNA-Prot | Missense |
| | | | Interaction | |

PD = Progressive Disease,
SD = Stable Disease,
PR = Partial Response

As used herein, the term “about” refers to plus or minus 10% of the referenced number. As used herein, the term “substantially similar to” means plus or minus 20% of the reference number. As used herein “about equal to” refers to plus or minus 5% of the reference number

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of treating a patient with a polypeptide comprising Ac-KPSSPPEE-NH₂ (SEQ ID NO:1), an active variant thereof, or a CD44-modulating peptide, wherein the patient has cancer, the method comprising:
a. determining a TP53 mutational status by:
i. obtaining or having obtained a biological sample from the patient; and
ii. performing or having performed a genotyping assay on the biological sample to determine the TP53 mutational status; and
b. administering a polypeptide comprising Ac-KPSSPPEE-NH₂ (SEQ ID NO:1), an active variant thereof, or a CD44-modulating peptide, to a patient if the TP53 mutation status predicts a p53 loss of activity.

2. The method of claim 1, wherein the polypeptide is not administered if the TP53 mutational status predicts no tumor p53 expression or TP53 loss of function.

3. A method of determining effectiveness of a polypeptide comprising Ac-KPSSPPEE-NH₂ (SEQ ID NO:1), an active variant thereof, or a CD44-modulating peptide to treat cancer in a patient, the method comprising:
a. determining a TP53 mutational status by:
i. obtaining or having obtained a biological sample from the patient; and
ii. performing or having performed a genotyping assay on the biological sample to determine the TP53 mutational status; and
b. determining the effectiveness of the polypeptide to treat cancer in the patient based on the TP53 mutational status,
wherein if the TP53 mutational status predicts a p53 loss of activity, the polypeptide is determined to be effective at treating cancer in the patient, and the polypeptide is administered to the patient; or if the TP53 mutational status predicts no tumor p53 expression or TP53 loss of function, the polypeptide is determined to not be effective at treating cancer in the patient, and the polypeptide is not administered to the patient.

4. The method of claim 1, wherein the cancer is a resistant or refractory cancer.

5. The method of claim 4, wherein the cancer is resistant or refractory to treatment with chemo-, immuno-, gene, and/or radiation therapy.

6. The method of claim 1, wherein the cancer is a solid tumor or hematological cancer.

7. The method of claim 1, wherein the cancer is selected from a group consisting of ovarian cancer, breast cancer, colorectal cancer, prostate cancer, head and neck cancer, endometrial cancer, primary peritoneal cancer, liver cancer, glioblastoma, or a combination thereof.

8. The method of claim 1, wherein the biological sample is selected from a group consisting of tissue, blood, and cells, saliva, and/or cerebrospinal fluid (CSF).

9. The method of claim 8, wherein the tissue further comprises fresh tissue, frozen tissue, and/or formalin-fixed, paraffin-embedded (FFPE).

10. The method of claim 1, wherein the TP53 mutational status comprises a mutation selected from the group consisting of: R342* (* mutation in oligomerization domain), D259V (non-functional protein), R175H, R273C, R213* (* truncated/nonsense), G105fs* (* truncated/frameshift), E286Q, C238W, and wildtype.

11. The method of claim 1, wherein the TP53 mutation status that predicts a partial response comprises mutations selected from the group consisting of R342*, R273C, and C238W.

12. The method of claim 1, wherein the TP53 mutation status that predicts a stable disease comprises mutations selected from the group consisting of R175H, E286Q, and no mutation (wildtype (WT)).

13. The method of claim 1, wherein the TP53 mutation status that predicts a progressive disease comprises mutations selected from the group consisting of D259V, R213*, and G105fs*.

14. The method of claim 1, wherein the CD44-modulating polypeptide is a polypeptide comprising SEQ ID NO:1 or SEQ ID NO:2.

15. The method of claim 1, wherein the CD44-modulating polypeptide comprises a variant sequence of SEQ ID NO:1, wherein the variant sequence comprises one or more amino acid mutation with respect to SEQ ID NO: 1 selected from:
a. K1 to A;
b. P2, P5, P6, or a combination thereof to A;
c. S3, S4, or S3 and S4 to A; or
d. E7, E8, or E7 and E8 to A,
wherein the mutation retains CD44-modulating activity about equal to or greater than a polypeptide of SEQ ID NO:1.

16. The method of claim 1, wherein the CD44-modulating polypeptide is administered at an amount of about 10 mg to about 600 mg.

17. The method of claim 1, wherein the CD44-modulating polypeptide is administered once a day (QD) or twice daily (BID).

18. The method of claim 1, wherein the CD44-modulating polypeptide is administered during the first period continuously and without a rest period.

19. The method of claim 1, wherein the administration of SEQ ID NO:1 is parenteral administration.

20. The method of claim 19, wherein the parenteral administration comprises subcutaneous administration, intradermal administration, and/or intranasal administration.
